(19) United States
(12) Patent Application Publication
(10) Pub. No.: US 2026/0026939 A1
(43) Pub. Date: Jan. 29, 2026

(54) ARTHROPLASTY FEMORAL IMPLANT DEVICES

(71) Applicants: Smith & Nephew, Inc., Memphis, TN (US); Smith & Nephew Orthopaedics AG, Zug (CH); Smith & Nephew Asia Pacific Pte. Limited, Singapore (SG)

(72) Inventors: Christopher R. Cyko, Bartlett, TN (US); Jason S. Jordan, Hernando, MS (US)

(73) Assignees: Smith & Nephew, Inc., Memphis, TN (US); Smith & Nephew Orthopaedics AG, Zug (CH); Smith & Nephew Asia Pacific Pte. Limited, Singapore (SG)

(21) Appl. No.: 18/994,237

(22) PCT Filed: Jul. 27, 2023
(86) PCT No.: PCT/US23/28841

§ 371 Date: Jan. 14, 2025

Related U.S. Application Data
(60) Provisional application No. 63/370,142, filed on Aug. 2, 2022.

(51) Int. Cl. A61F 2/38; A61F 2/30

(52) U.S. Cl. CPC A61F 2/3859; A61F 2002/30327; A61F 2002/3895

(57) ABSTRACT

Disclosed herein is a femoral component (100) of a knee arthroplasty system. The femoral component may be a multi-radius (MR) component having multiple centers of rotation within the functional knee range of motion. The femoral component may include at least two of a mid-flexion radius (141), an extension radius (142), or a deep flexion radius (140). The femoral component may include an articulate surface (112) that is handed to conform to a medial condyle, a lateral condyle, a right knee, a left knee, and/or the like. The femoral component may be configured based on, among other things, a relationship between a mid-flexion region (131) and an AP dwell (“MFR/AP relationship”). Accordingly, the femoral component may be in the form of a MR component with a spherical or substantially spherical mid-flexion region in a handed configuration. The femoral component may be configured as a unicompartmental femoral implant or a total knee arthroplasty (TKA) femoral implant.

### CROSS-REFERENCE TO RELATED APPLICATION

This application claims the benefit of U.S. Provisional Patent Application Ser. No. 63/370,142, filed Aug. 2, 2022, and titled “Arthroplasty Femoral Implant Devices,” the entire contents of which application is hereby incorporated by reference in its entirety.

### TECHNICAL FIELD

The present disclosure is directed to orthopedic implants, and more specifically to a multi-radius femoral component of a knee arthroplasty implant system.

### BACKGROUND

The biomechanics of the knee are highly complex and influenced by multiple anatomical characteristics. Significant disease or injury affecting the knee joint may be treated by a knee arthroplasty procedure to surgically replace the ends, or a portion thereof, of the femur and/or tibia with a prosthetic knee device that may include a prosthetic femoral implant and/or a tibial implant. A knee arthroplasty (KA) procedure may be a partial (unicondylar or unicompartmental knee arthroplasty (UKA)) procedure or a total knee arthroplasty (TKA) procedure. UKA is a surgical technique used for the treatment of one compartment of the knee (i.e., the medial compartment or the lateral compartment). In contrast, a TKA is used for the treatment of all three compartments of the knee (i.e., the medial, lateral, and patellofemoral compartments).

During a knee replacement procedure, the femoral implant may be placed on a patient's distal femur (or portion thereof, such as to replace a medial or lateral condyle in a UKA procedure) after appropriate resection of the femur. The tibial implant may include a tibial tray that generally conforms to a resected proximal tibia. The femoral implant may be configured to engage or articulate against a corresponding surface of the tibial implant.

A common complaint of KA patients is that the replaced knee does not function or feel like a normal knee. Trade-offs between functionality and stability cause conventional prosthetic knee devices to produce kinematics different than the normal knee. For example, patient flexion and/or extension of the knee while walking may be limited. In more serious cases, pain or discomfort may occur during articulation of the prosthetic knee device.

Accordingly, knee arthroplasty patients would benefit from prosthetic devices that support natural operation of the knee, including facilitating component range of motion to alleviate the pain and instability associated with conventional prosthetic knee devices, while also providing sufficient strength and stability to avoid implant failure.

### SUMMARY

This Summary is provided to introduce a selection of concepts in a simplified form that are further described below in the Detailed Description. This Summary is not intended to identify key features or essential features of the claimed subject matter, nor is it intended as an aid in determining the scope of the claimed subject matter.

The present disclosure describes a femoral component of a prosthetic knee implant system. In any preceding or subsequent examples, the femoral component may be a unicompartmental implant for a unicompartmental knee arthroplasty (UKA) procedure. A unicompartmental femoral component may be configured for either a medial condyle or a lateral condyle. In any preceding or subsequent examples, a unicompartmental femoral component may be non-symmetric (or “handed”), for example, having a directionality, curvature, and/or the like of at least a portion thereof (for instance, a portion of an articular surface) to conform to a particular portion of anatomy. For example, a unicompartmental femoral component may be handed to conform to a medial condyle, a lateral condyle, a right knee, a left knee, and/or the like.

Although a unicompartmental femoral implant is used in examples of femoral components in the present disclosure, examples are not so limited, for instance, the femoral components may include a femoral component for a total knee arthroplasty (TKA) procedure. In addition, examples described with respect to unicompartmental femoral implants may be configured for a TKA femoral component. Accordingly, a femoral component described in the present disclosure may refer to either a unicompartmental femoral component or a TKA femoral component unless specified otherwise.

The femoral component according to various examples may include an articular surface configured to conform or substantially conform to the native anatomical surface of patient anatomy. In any preceding or subsequent examples, the femoral component may include a multi-radius (MR) configuration having multiple centers of rotation within the functional knee range of motion. The multiple centers of rotation may be facilitated by changing radii (radii of curvature or arc radii; terms used interchangeably herein without the intent to limit), and therefore, changing curvature of the articular surface, of the femoral component. In any preceding or subsequent examples, the femoral component may include at least two of a mid-flexion radius, an extension radius, or a deep flexion radius.

In any preceding or subsequent examples, the femoral implant may include an articular surface featuring a spherical cross section in a mid-flexion region (for instance, from approximately 15-90 degrees of flexion). In any preceding or subsequent examples, the femoral implant may include a larger radius (i.e., larger than the mid-flexion region radius or radii) or multiple larger sagittal radii in an extension area (for instance, less than approximately 15 degrees of flexion). In any preceding or subsequent examples, the femoral implant may include a smaller sagittal radius (i.e., smaller than the mid-flexion region radius or radii) or multiple smaller radii in the deep flexion area (above approximately 90 degrees of flexion).

Accordingly, in any preceding or subsequent examples, the femoral component may be in the form of a MR component with a spherical or substantially spherical mid-flexion region in a handed configuration.

In any preceding or subsequent examples, the mid-flexion (or posterior) radius may be about 15 mm to about 25 mm. In any preceding or subsequent examples, the deep flexion (or distal) radius may be about 25 mm to about 50 mm. In any preceding or subsequent examples, the extension radius may be about 30 mm to about 70 mm.

In any preceding or subsequent examples, the mid-flexion radius may span about 15 degrees to about 90 degrees of flexion. In any preceding or subsequent examples, the deep flexion radius may span about 90 degrees of flexion to a maximum degree of flexion (for instance, about 150 degrees of flexion). In any preceding or subsequent examples, the extension radius may span from about 0 degrees of flexion to about 15 degrees of flexion.

In any preceding or subsequent examples, the femoral component may be configured with an anteroposterior (AP) length that is twice, substantially twice, and/or approximately twice the radial length of the mid-flexion region.

In any preceding or subsequent examples, a femoral component may be configured based on, among other things, a relationship between a mid-flexion region (MFR) and an AP dwell (“MFR/AP relationship”).

In any preceding or subsequent examples, the MFR/AP relationship may be determined according to the following: AP dwell=<value 1>(mid-flexion radius)−<value 2>. In any preceding or subsequent examples, value 1 may be about 1.0 to about 2.0. In any preceding or subsequent examples, value 2 may be about 2.0 to about 7.0.

In any preceding or subsequent examples, a femoral component may include a flexion curve transition (for example, between a deep flexion arc of the articulate surface defined by a deep flexion radius and a mid-flexion arc of the articulate surface defined by a mid-flexion radius) arranged between about 85 degrees of flexion to about 110 degrees of flexion.

In any preceding or subsequent examples, a femoral component may include an extension curve transition (for example, between an extension arc of the articulate surface defined by an extension radius and a mid-flexion arc of the articulate surface defined by a mid-flexion radius) arranged between about 5 degrees of extension to about 30 degrees of flexion.

In any preceding or subsequent examples, the femoral component may be configured with a transition between radii (for instance, a reduction in size between radii) that is about 0.5 millimeters (mm), about 1 mm, about 1.5 mm, about 2.0 mm, about 3.0 mm, and any value or range between any two of these values (including endpoints).

In any preceding or subsequent examples, the femoral component may include one or two pegs for affixing the femoral component to the distal portion of the femur.

In any preceding or subsequent examples, the femoral component may be arranged and configured to be used within a computer-assisted surgery (CAS) system or an orthopedic robotic surgical system.

In any preceding or subsequent example, a multi-radius (MR) femoral component of a knee arthroplasty system may include an articular surface configured to engage a corresponding tibial component, the articular surface comprising a plurality of regions, each of the plurality of regions associated with a different knee flexion/extension region and defined by one of a plurality of radii of curvature of the articular surface, wherein the plurality of radii comprises a deep flexion radius, a mid-flexion radius, and an extension radius.

In any preceding or subsequent example of the MR femoral component, the deep flexion radius is configured for greater than about 90 degrees of flexion.

In any preceding or subsequent example of the MR femoral component, the mid-flexion radius is configured for about 15 degrees to about 90 degrees of flexion.

In any preceding or subsequent example of the MR femoral component, the extension radius is configured for less than about 15 degrees of flexion.

In any preceding or subsequent example of the MR femoral component, the articular surface is non-faceted at transitions between the plurality of regions.

In any preceding or subsequent example of the MR femoral component, the mid-flexion radius has a circumference of about 15 mm to about 25 mm.

In any preceding or subsequent example of the MR femoral component, the deep flexion radius has a circumference of about 25 mm to about 50 mm.

In any preceding or subsequent example of the MR femoral component, the extension radius has a circumference of about 30 mm to about 70 mm.

In any preceding or subsequent example of the MR femoral component, a transition between at least two of the plurality of radii is about 0.5 mm to about 3.0 mm.

In any preceding or subsequent example of the MR femoral component, at least one of the plurality of radii are formed of a plurality of sub-radii.

In any preceding or subsequent example of the MR femoral component, the articular surface is configured according to a mid-flexion radius (MFR)/anteroposterior (AP) dwell relationship.

In any preceding or subsequent example of the MR femoral component, the MFR/AP relationship is defined by: AP dwell=<MFR factor>(MFR)−<offset value>.

In any preceding or subsequent example of the MR femoral component, the MFR factor is a value from about 1.0 to about 2.0.

In any preceding or subsequent example of the MR femoral component, the offset value is a value from about 2.0 to about 7.0.

In any preceding or subsequent example of the MR femoral component, an optimal MFR/AP relationship is defined by: AP dwell=<1.4015>(MFR)−<5.6611>.

In any preceding or subsequent example of the MR femoral component, an optimal MFR/AP relationship is defined within the following first bounding curve and second bounding curve: AP dwell_{first bounding curve}=<1.4493>(MFR)−5.4581 and AP dwell_{second bounding curve}=<1.1636>(MFR)−<2.4561>.

In any preceding or subsequent example of the MR femoral component, the MR femoral component is non-symmetric to conform to one of a medial condyle or a lateral condyle.

In any preceding or subsequent example of the MR femoral component, the MR femoral component is a unicompartmental femoral implant.

In any preceding or subsequent example, a femoral component of a knee arthroplasty system may include an articular surface configured to engage a corresponding tibial component, the articular surface comprising a mid-flexion region defined by a mid-flexion radius (MFR), wherein the articular surface is configured according to a mid-flexion radius (MFR)/anteroposterior (AP) dwell relationship.

In any preceding or subsequent example of the femoral component, the MFR/AP relationship is defined by: AP dwell=<MFR factor>(MFR)−<offset value>.

In any preceding or subsequent example of the femoral component, the MFR factor is a value from about 1.0 to about 2.0.

In any preceding or subsequent example of the femoral component, the offset value is a value from about 2.0 to about 7.0.

In any preceding or subsequent example of the femoral component, an optimal MFR/AP relationship is defined by: AP dwell=<1.4015>(MFR)−<5.6611>.

In any preceding or subsequent example of the femoral component, an optimal MFR/AP relationship is defined within the following first bounding curve and second bounding curve: AP dwell_{first bounding curve}=<1.4493>(MFR)−5.4581 and AP dwell_{second bounding curve}=<1.1636>(MFR)−<2.4561>.

In any preceding or subsequent example of the femoral component, the articular surface comprises a plurality of regions, each of the plurality of regions associated with a different knee flexion/extension region and defined by one of a plurality of radii of curvature of the articular surface, wherein the plurality of radii comprises a deep flexion radius, the MFR, and an extension radius.

In any preceding or subsequent example of the femoral component, the deep flexion radius is configured for greater than about 90 degrees of flexion.

In any preceding or subsequent example of the femoral component, the mid-flexion radius is configured for about 15 degrees to about 90 degrees of flexion.

In any preceding or subsequent example of the femoral component, the extension radius is configured for less than about 15 degrees of flexion.

In any preceding or subsequent example of the femoral component, the articular surface is non-faceted at transitions between the plurality of regions.

In any preceding or subsequent example of the femoral component, the mid-flexion radius has a circumference of about 15 mm to about 25 mm.

In any preceding or subsequent example of the femoral component, the deep flexion radius has a circumference of about 25 mm to about 50 mm.

In any preceding or subsequent example of the femoral component, the extension radius has a circumference of about 30 mm to about 70 mm.

In any preceding or subsequent example of the femoral component, a transition between at least two of the plurality of radii is about 0.5 mm to about 3.0 mm.

In any preceding or subsequent example of the femoral component, at least one of the plurality of radii are formed of a plurality of sub-radii.

In any preceding or subsequent example of the femoral component, the MR femoral component is non-symmetric to conform to one of a medial condyle or a lateral condyle.

In any preceding or subsequent example of the femoral component, the MR femoral component is a unicompartmental femoral implant.

Examples of the present disclosure provide numerous technological features and advantages over conventional systems. For example, advancements in knee instrument technology, specifically handheld robotics, allow additional intraoperative flexibility to identify, align, and prepare a patient's native anatomy to receive an implant, such as an implant configured according to some examples, with more precise and personalized features, such as an articular surface that more closely resembles native patient femur anatomy. Femoral components according to some examples may feature an articular surface designed to better match the native anatomical surface, offered in a handed design such that appropriate bony coverage may be maintained without compromise to the rotational placement of the implant. Additional advantages may include a bone facing surface mimicking the articular surface such that minimal bone removal is required to maintain strength. Application of the three-dimensional curvature to the bone facing side of the femoral component may also reduce the need for areas of artificial thickness or thinness, for example, found in designs with multiple planar faces. In addition, femoral components according to some examples may not require an additional keel, spar, or similar feature to maintain strength, which may compromise internal stress uniformity within the post operative patient anatomy; rather only two pegs may be sufficient for location and rotational fixation.

Accordingly, as described in more detail in the present disclosure, some examples may provide a femoral component formed with a geometry configured to provide improved implant stability and kinematic performance throughout the complete range of motion of the affected knee, including, within flexion, mid-flexion, and extension regions, when compared with conventional implant systems.

Examples are not limited in this context. Additional technological advantages and technical features over conventional systems would be known to those of skill in the art based on the present disclosure.

Further features and advantages of at least some of the examples of the present disclosure, as well as the structure and operation of various examples of the present disclosure, are described in detail below with reference to the accompanying drawings.

### BRIEF DESCRIPTION OF THE DRAWINGS

By way of illustration, various examples of disclosed devices will now be described, with reference to the accompanying drawings, in which:

FIG. 1A depicts a perspective view of an illustrative example of a femoral component of a unicompartmental implant system in accordance with the present disclosure;

FIG. 1B depicts a back or anterior view of the illustrative femoral component shown in FIG. 1A;

FIG. 1C depicts a bottom or distal view of the illustrative femoral component shown in FIG. 1A;

FIG. 1D depicts a side view of the illustrative femoral component shown in FIG. 1A;

FIG. 2 depicts a unicompartmental knee implant system in accordance with the present disclosure;

FIGS. 3A and 3B depict a side view of an illustrative example of a femoral component in accordance with the present disclosure;

FIG. 4 depicts a diagram of an illustrative example of a femoral component in accordance with the present disclosure;

FIG. 5 depicts diagrams of variability boundaries of an illustrative example of a femoral component in accordance with the present disclosure;

FIG. 6 depicts diagrams of various deep flexion curve transition posterior radius dimensions of an illustrative example of a femoral component in accordance with the present disclosure;

FIG. 7 depicts diagrams of various extension curve transition dimensions of an illustrative example of a femoral component in accordance with the present disclosure;

FIG. 8 depicts anteroposterior (AP) and radius relationship information associated with some examples of a femoral component in accordance with the present disclosure; and

FIGS. 9-12 depict kinematic information associated with some examples of a femoral component in accordance with the present disclosure.

It should be understood that the drawings are not necessarily to scale and that the disclosed examples are sometimes illustrated diagrammatically and in partial views. In certain instances, details which are not necessary for an understanding of the disclosed methods and devices, or which render other details difficult to perceive may have been omitted. It should be further understood that this disclosure is not limited to the particular examples illustrated herein. In the drawings, like numbers refer to like elements throughout unless otherwise noted.

### DETAILED DESCRIPTION

Various features of an improved femoral component of a knee implant system will now be described more fully hereinafter with reference to the accompanying drawings, in which one or more features of the femoral component will be shown and described. It should be appreciated that the various features may be used independently of, or in combination, with each other. It will be appreciated that a femoral component as disclosed herein may be embodied in many different forms and should not be construed as being limited to the examples set forth herein. Rather, these examples are provided so that this disclosure will convey certain features of the femoral component to those skilled in the art.

Knee prostheses are developed with several competing design factors, including, for instance, instrumentation, stability, fixation, and/or functional kinematics of the knee. In addition, for a unicompartmental knee arthroplasty (UKA) system, another design factor may include ease of revision to a total knee arthroplasty (TKA) responsive to UKA failure. It is challenging to achieve such a diverse number of competing design goals in a single device. Accordingly, conventional knee implants are constructed with trade-offs or compromises between design goals, such as reduced kinematic performance for increased stability, and/or the like.

For example, a typical compromise has been to use an articular shape that is effective for a three-planar resection surgical technique and ease of revision, which results in an articular shape that features a smaller articular radius (for instance, when compared to native anatomy) in the mid-flexion region. As a result, patients may typically experience mid-flexion tightness, particularly without modification of the implantation surgical technique to replace a similar amount of bone to the amount that was removed.

In another example, a compromise is to use a femoral implant with a single radius (SR), for instance, specified for the mid-flexion region. The radius is determined based on a direct or indirect measurement of the mid-flexion region of the patient, resulting in a spherical implant based on this measured feature. Such spherical implants may target mid-flexion performance goals; however, spherical implants are generally mismatched in the areas of articulation outside of the mid-flexion region (i.e., extension and deep flexion). Accordingly, SR designs are typically optimized for one particular region, such as the mid-flexion region, but are deficient in other areas, including, for example, failing to provide an anatomic anteroposterior (AP) extension dwell position, an undersized radius in extension, and/or oversized in deep flexion.

In a further example, multi-radius (MR) (or “J-curve”) femoral implants have been used that attempt to provide better correspondence with native anatomy in the various flexion/extension regions, for instance, when compared with a spherical implant. However, conventional MR implants have multiple deficiencies. For instance, conventional MR implants have been designed in symmetric (non-“handed”) configurations that require them to be implanted with axial inclination, which ultimately changes the articular track with respect to native anatomy. In addition, conventional MR implants may cause knee strain in certain regions of flexion/extension, for instance, medial collateral ligament (MCL) strain that may lead to patient discomfort or even injury.

Conventional MR designs typically exhibit a late transition from the extension to flexion radii and undersized flexion radii to maintain balance between AP dwell location, characteristics that allow for ease of revision to TKA such as minimized implant thicknesses, and/or a minimized superoinferior (SI) height due to disconnect between anatomic articular and sizing (for instance, sizing may occur via making arbitrary resections then matching AP length of uncovered bone or transposing anatomic mark from tibia then matching AP length). Conventional MR designs are typically implanted anterior (for instance, about 2 mm anterior) with additional bone removed for an instrumented implantation or with a change in flexion angle with a computer-assisted or robotic implantation process (for instance, to overcome MCL strain in the mid-flexion region).

Accordingly, as described in more detail in the present disclosure, some examples may provide a femoral component formed with a geometry configured to provide improved implant stability and kinematic performance throughout the complete range of motion of the affected knee, including, within flexion, mid-flexion, and extension regions, when compared with conventional implant systems.

As will be described herein, an implant device, including a femoral component thereof, may have various shapes, sizes, and/or configurations of other physical properties. It should be appreciated that the implant device may be provided in any suitable shape and/or configuration, which, as will be appreciated by one of ordinary skill in the art, may be dependent on the location and type of patient bone being fixed. For example, an implant device may include various bone conforming segments configured to correspond with different anatomical features and/or prepared portions of patient anatomy. In addition, the femoral component may be a part of an implant device arranged and configured to span, contact, be affixed to, and/or the like various portions of a human knee, including without limitation, the tibia and/or femur.

A femoral component according to some examples may include any now known or hereafter developed additional features. The femoral component may be manufactured from any suitable material now known or hereafter developed, including, for example, metals, polymers, plastics, ceramics, resorbable, non-resorbable, composite materials, and/or the like. Suitable materials may include, without limitation, titanium, stainless steel, cobalt chrome, polyetheretherketone (PEEK), polyethylene, ultra-high molecular weight polyethylene (UHMWPE), resorbable polylactic acid (PLA), polyglycolic acid (PGA), combinations or alloys of such materials or any other appropriate material that has sufficient strength to be secured to and hold bone, while also having sufficient biocompatibility to be implanted into a human body.

Although unicompartmental femoral components are used in some examples in the present disclosure, examples are not so limited. Therefore, it should be appreciated that the present disclosure should not be limited to any particular configuration of implant device and/or insertion procedure unless specifically claimed. In addition, while the present disclosure will be described and shown as being directed to a unicompartmental femoral implant, it should be appreciated that features of the present disclosure have applicability and may be used in connection with other implant devices such as, a TKA implant system, and/or the like.

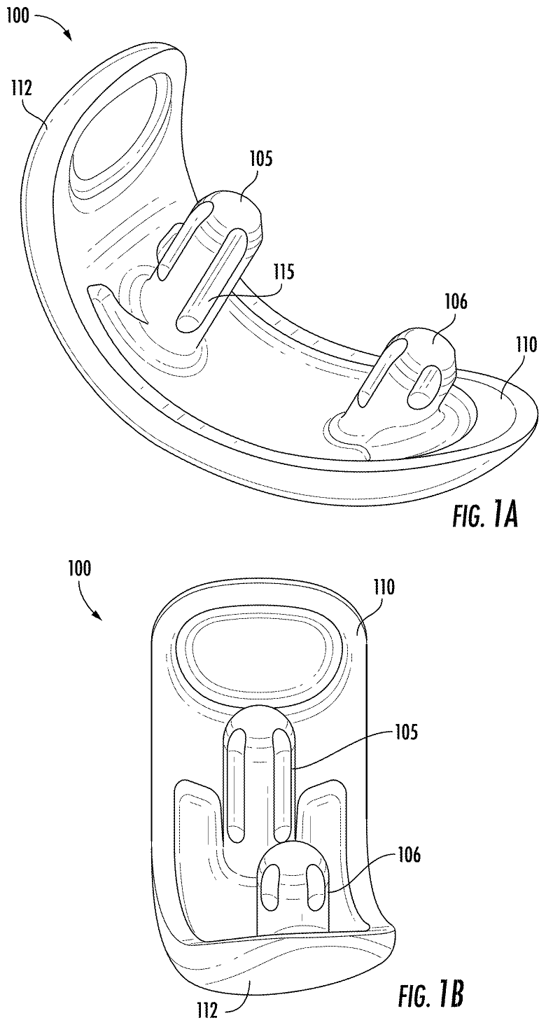

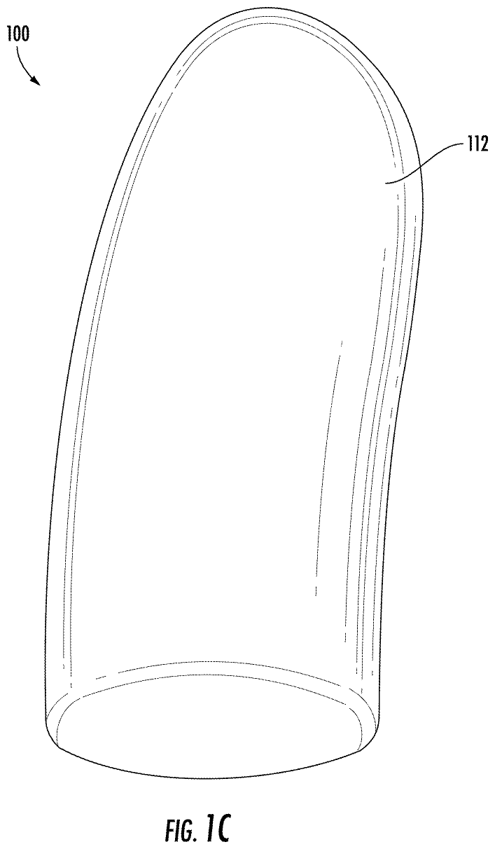

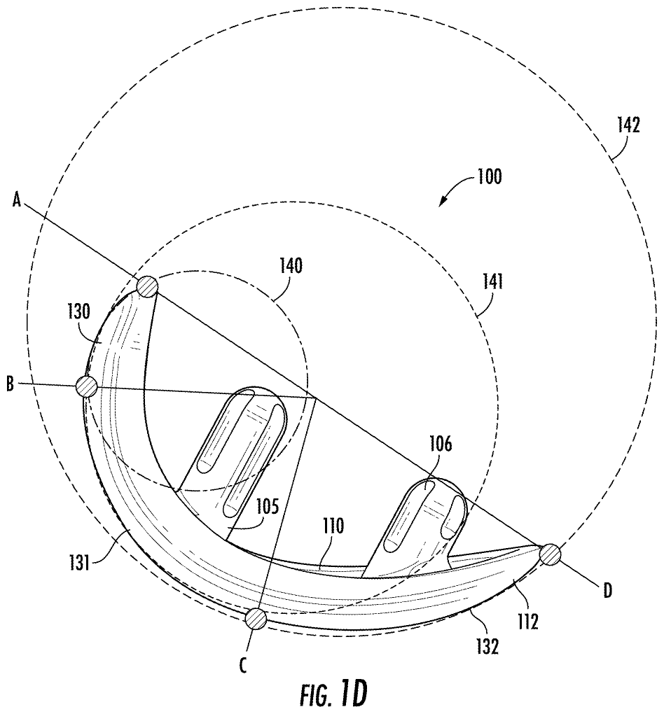

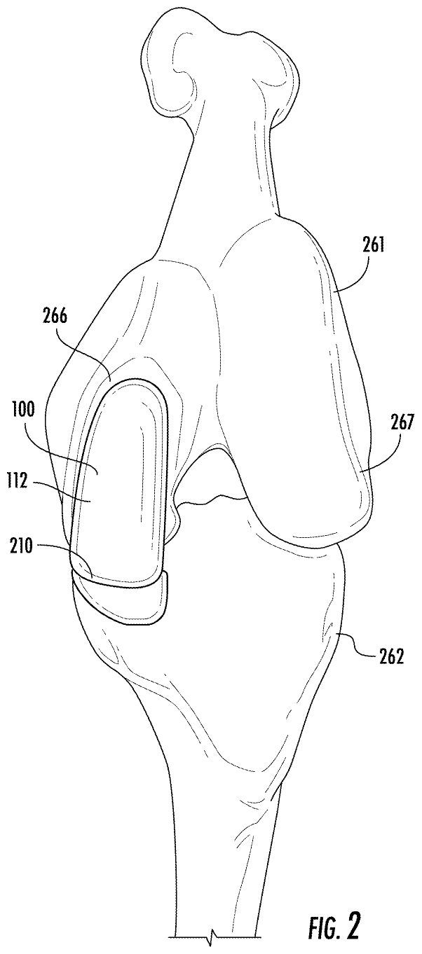

FIG. 1A depicts a perspective view of an illustrative example of a femoral component in accordance with the present disclosure. FIG. 1B depicts a back or anterior view of the illustrative femoral component shown in FIG. TA. As shown in FIGS. TA and 1B, a femoral component 100 may include an inner surface 110 configured to engage, contact, be mounted against, and/or the like a prepared distal portion of a femur.

For example, FIG. 2 depicts a unicompartmental knee implant system in accordance with the present disclosure implanted within example patient anatomy. As shown in FIG. 2, femoral component 100 may be implanted on/within a prepared surface of a medial condyle 266 of a femur 261. Although femoral component 100 is shown in FIG. 2 as being implanted on/within medial condyle 266, femoral component 100 may be configured for implantation on/within a lateral condyle 267. Femoral component 100 may include an articular surface 112 that faces away from femur 261 and is configured to face and engage or articulate against a tibial implant 210 implanted on a prepared surface of a tibia 262.

Referring to FIGS. 1A and 1B, in some examples, femoral component 100 may include one or more pegs 105, 106 protruding from inner surface 110. In general, pegs 105, 106 may be configured to be inserted within corresponding cavities created within a prepared surface of a femur to facilitate affixing femoral component 100 to the femur. In various examples, femoral component 100 may have two pegs 105, 106. In any preceding or subsequent examples, pegs 105, 106 may include certain mounting features 115 to support affixation of femoral component to a femur, for instance, via facilitating bone growth in/around pegs 105, 106, application of cement or another adhesive, and/or protruding into an inner wall of the cavities. Non-limiting examples of mounting features 115 may include grooves, bumps, protrusions, holes, and/or the like.

In some examples, femoral component 100 may be a cementless implant component configured for cementless fixation to a femur. In a cementless (interference-fit or press-fit) configuration, femoral component 100 uses friction forces to form an interference fit between the implant component and the prepared bone. Subsequent bone growth facilitates osseointegration to fixate the implant with the bone. In a cementless configuration, femoral component 100 may include various fixation members, including, for instance, pegs 105, 106 and/or posts, fins, rails, cavities, anchors, protrusions, concave surfaces, convex surfaces, grooves, projections, bumps, and/or a combination thereof.

As shown in FIG. 1A, femoral component 100 may include an articular surface 112 arranged on an opposite side of femoral component 100 from inner surface 110. In general, articular surface 112 may be configured to engage or articulate against a tibia or tibial component (not shown; see FIG. 2) of a knee implant system.

Advancements in knee instrument technology, specifically handheld robotics, allow additional intraoperative flexibility to identify, align, and prepare a patient native anatomy to receive a knee implant, including an improved femoral component configured according to some examples. For example, a challenge with unicompartmental implants is the technical difficulty of implantation in the patient. Using conventional, instrumented techniques, planar cuts were made to the patient anatomy and the implant was chosen, sized, shaped, etc. based on how well it matched the planar cuts. This process was largely decoupled from the kinematics of the implant. By matching the planar cuts, the articulating surface was essentially ignored, resulting in implants that did not closely correspond to native patient anatomy. For SR implants, to get the implant to stay in place, surgeons would target the mid-flexion area and match it. Regions (for instance, deep flexion and extension) were ignored because the focus was on keeping the implant in place. As a result, the kinematic performance and stability of the implant was compromised.

In various examples, all or a portion of a knee arthroplasty process using a femoral component according to some examples may be performed by a computer-implemented method via specifically programmed computer hardware and/or software. For example, all or a portion of a knee arthroplasty process using a femoral component according to some examples may be performed by a computer-assisted surgery (CAS) system for performing computer- and/or robotic-assisted surgery on a patient. Using CAS and robotic systems, surgeons may focus more on the kinematics of an implant, such as femoral components according to some examples. As a result, the implant components may define the bone contacting surface and the CAS or robotic systems can prepare patient anatomy match it.

In view of the available flexibility and precision bone cutting/preparation, femoral components according to some examples may feature an articular surface designed to more precisely and accurately match the native anatomical surface of a patient knee, configured in a non-symmetrical (or “handed”) design (see, for example, FIG. 1C) such that appropriate bony coverage may be maintained without, as opposed to conventional implants (including existing MR femoral components), compromising the rotational placement of the implant. In addition, the handed configuration may operate to minimize mismatch occurring from axial rotational placement with an AP length, for instance, roughly twice the length of the mid-flexion radius, for adequate placement between a native posterior femur and a line projected onto the femur from the native tibia in terminal extension (i.e., a tide mark).

Additional improvements over conventional implant devices may include a bone facing surface that corresponds or substantially corresponds (for instance, mimicking or mirroring) the articular surface of native femoral anatomy such that minimal bone removal is required to maintain strength. Application of this three-dimensional (3D) curvature to the bone facing side of the implant may also reduce the need for areas of artificial thickness or thinness found in conventional MR designs, for example, with multiple planar faces. Moreover, femoral component 100 may rely on two pegs 105, 106, instead of requiring additional keel or spar features to maintain strength, which may compromise internal stress uniformity within the post-operative patient anatomy. Examples are not limited in this context. Additional improvements and technical features would be known to those of skill in the art based on the present disclosure.

FIG. 1C depicts a bottom or distal view of the illustrative femoral component shown in FIG. TA. As shown in FIG. 1C, articular surface 112 of femoral component 100 may be asymmetrical (or “handed”). Femoral component 100 may be specifically designed for a left knee or right knee. For a UKA implant system, in addition or in the alternative, femoral component 100 may be specifically designed for a medial condyle or a lateral condyle. Accordingly, articular surface 112 may be asymmetrical to match, mimic, or otherwise correspond to native patient anatomy.

For example, articular surface 112 may be handed to correspond with the native patient anatomy of a left knee, a right knee, a medial condyle, a lateral condyle, a medial condyle of the left knee, a medial condyle of the right knee, a lateral condyle of the left knee, a lateral condyle of the right knee. Examples are not limited in this context.

FIG. 1D depicts a side view of the illustrative femoral component shown in FIG. TA. As shown in FIG. 1D, femoral component 100 may be a MR femoral component. In general, a “radius” may be defined as a distance from the flexion/extension axis of rotation to the contact point between the femoral and tibial components of the implant.

In various examples, femoral component 100 may be configured for radii of rotation that generally correspond with associated regions of flexion/extension of the knee. For example, in some examples, femoral component 100 may be configured with at least two of a deep flexion radius 140 (greater than about 90 degrees of flexion), a mid-flexion radius 141 (from about 15 degrees to about 90 degrees of flexion), and an extension radius (less than about 15 degrees of flexion) 142. In various examples, mid-flexion radius 141 may cause articulate surface 112 to have a mid-flexion region that is spherical or substantially spherical. In any preceding or subsequent examples, each of radii 140, 141, and 142 may include one or more sub-radii, for example, a radius may include multiple smaller radii in a certain region (for instance, to provide different articular surface geometries).

The multiple radii 140, 141, and/or 142 of femoral component 100 causes articular surface 112 to have different curve geometries within different flexion/extension regions. For example, femoral component 100 may include a deep flexion region 130 (generally, an arc of articular surface 112 between points A and B), a mid-flexion region 131 (generally, an arc of articular surface 112 between points B and C), and an extension region 132 (generally, an arc of articular surface 112 between lines C and D). An articular surface 112 with different regions or arcs 130, 131, and/or 132 may facilitate femoral component 100 being more closely aligned with native patient anatomy and, therefore, providing patients with improved implant stability and native knee kinematics.

Some examples may be configured with different dimensional changes between radii 140-142. The different dimensional changes may affect the geometry, transition, and/or the like between different regions or arcs 130, 131, and/or 132. In some examples, the radii may be increasing in size following articulate surface 112 from deep flexion region 130 to extension region 132. For example, a difference between radii 140 and 141 (for instance, (circumference of radius 140)−(circumference of radius 141)) may be about 2 mm. In various examples, a difference between radii 140 and 141 may be about 2.0 mm, about 2.5 mm, about 3.0 mm, about 3.5 mm, about 5.0 mm and any value or range between any two of these values (including endpoints).

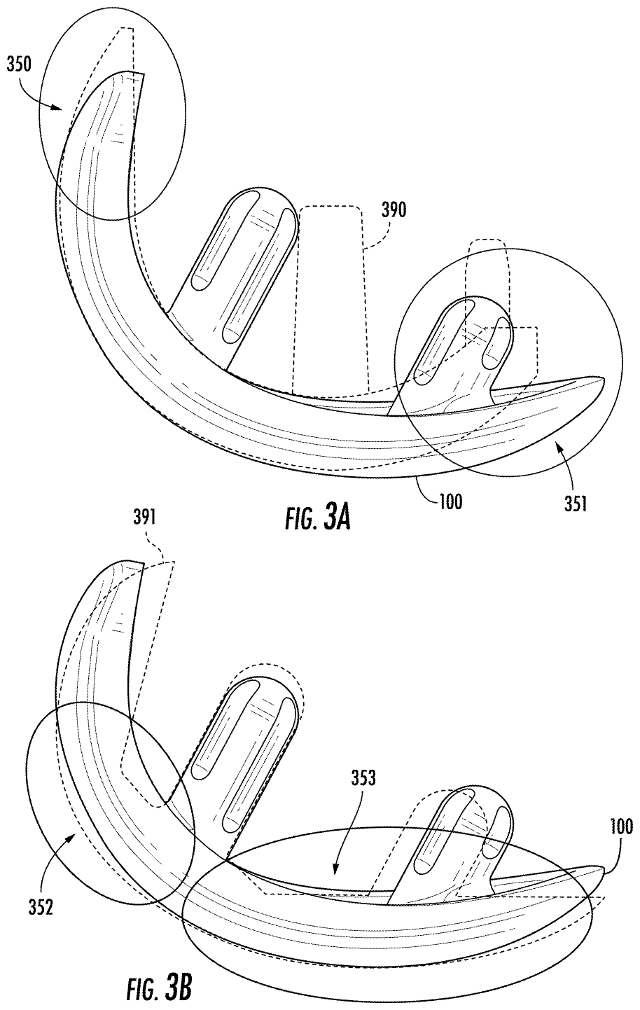

FIGS. 3A and 3B depict a side view of an illustrative example of a femoral component in accordance with the present disclosure. More specifically, FIGS. 3A and 3B show a femoral component 100 configured according to some examples overlayed on a conventional femoral component 390 (FIG. 3A) and 391 (FIG. 3B).

Referring to FIG. 3A, conventional femoral component 390 may be the same or similar to a spherical femoral component. A non-limiting example of a spherical femoral component may be the same or similar to femoral components described in U.S. Pat. No. 5,314,482, titled “Femoral Component, Tool and Method.” Another non-limiting example of a femoral component corresponding to conventional femoral component 390 may be the femoral component of the Oxford® unicompartmental knee system provided by Zimmer Biomet located in Warsaw, Indiana, United States. A further non-limiting example of a femoral component corresponding to conventional femoral component 390 may be the femoral component of the Uniglide™ unicompartmental knee system provided by the Corin Group located in Cirencester, United Kingdom. Femoral component 390 may most closely resemble an Oxford® femoral component.

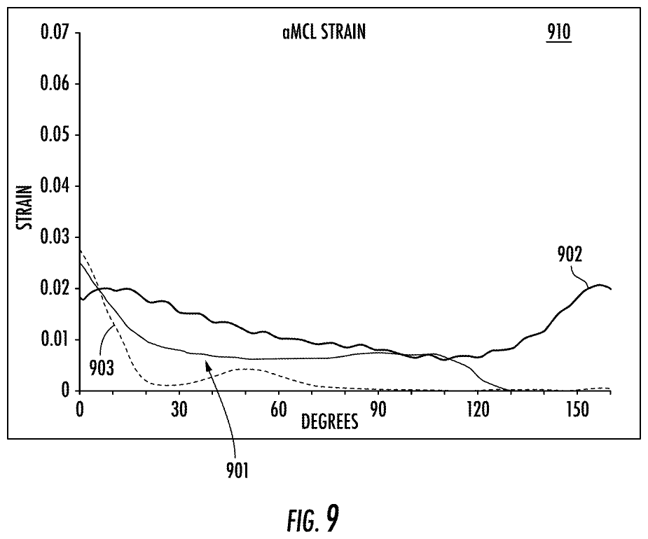

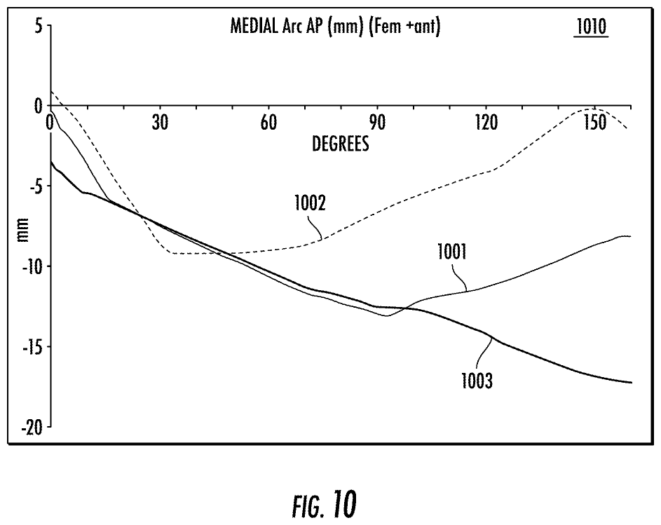

As shown in FIG. 3A, femoral component 100 may provide improvements in geometry over femoral component 390 in at least a deep flexion region 350 and an extension region 351. In addition, conventional femoral components, such as femoral component 390 are not asymmetrical (or “handed”). Accordingly, femoral component 390 is not able to provide the kinematic performance provided by femoral component 100 configured according to some examples (see, for example, FIGS. 9-12).

Although improvements in a deep flexion region 350 and an extension region 351 are specifically referenced in FIG. 3A, a person of skill in the art would recognize that femoral component 100 may provide additional technological advantages over conventional femoral components, such as femoral component 390.

Referring to FIG. 3B, conventional femoral component 391 may be the same or similar to a traditional femoral component. A non-limiting example of a femoral component corresponding to conventional femoral component 391 may be the femoral component of the Mako® unicompartmental knee system provided by Stryker located in Kalamazoo, Michigan, United States.

As shown in FIG. 3B, femoral component 100 may provide improvements in geometry over femoral component 391 in at least a mid-deep flexion region 352 and in component thickness 353. Accordingly, femoral component 391 is not able to provide the kinematic performance provided by femoral component 100 configured according to some examples (see, for example, FIGS. 9-12).

Although improvements in a mid-deep flexion region 352 and in component thickness 353 are specifically referenced in FIG. 3B, a person of skill in the art would recognize that femoral component 100 may provide additional technological advantages over conventional femoral components, such as femoral component 391.

As indicated in FIGS. 3A and 3B, in some examples, femoral component 100 may provide, inter alia, improved mid-flexion performance in a handed configuration. As a result, femoral component 100 may provide improved kinematic performance over existing femoral component designs, including spherical, SR, MR, and/or other traditional components (see, for example, FIGS. 9-12).

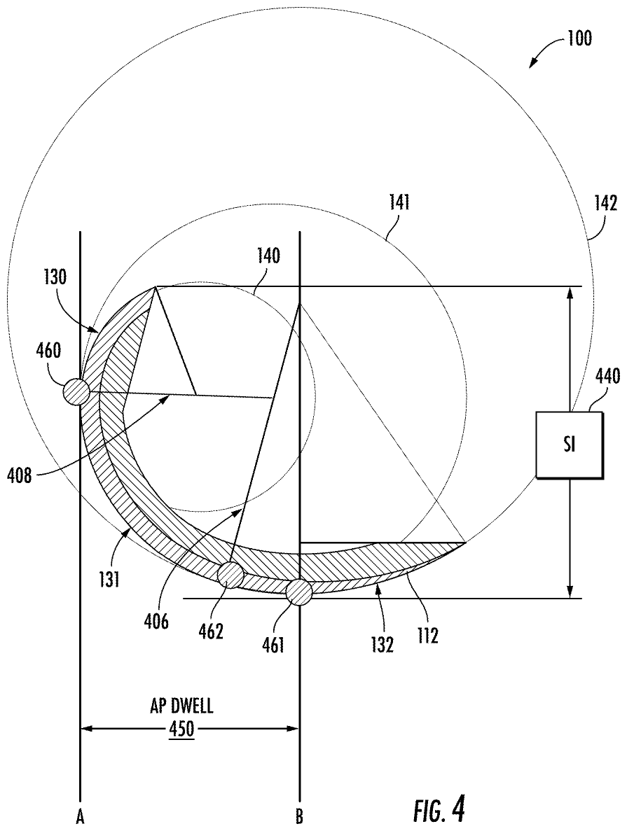

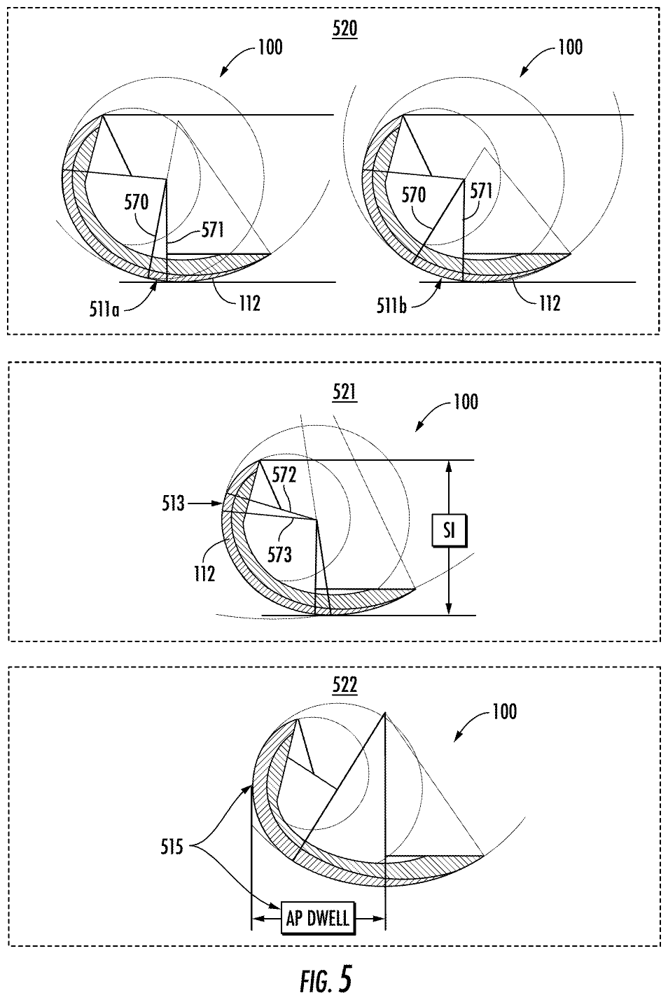

FIG. 4 depicts a diagram of an illustrative example of a femoral component in accordance with the present disclosure. As shown in FIG. 4, a femoral component 100 configured according to some examples may include various features, including technological features that provide multiple advantages over conventional knee implant systems. The features of femoral component 100 may operate in combination to provide improved stability and/or kinematic performance for a knee implant system using a femoral component configured according to some examples.

As shown in FIG. 4, femoral component 100 may be a MR component, having two or more of radii 140-142. In general, radius 140 may be for deep flexion, radius 141 may be for mid-flexion, and radius 142 may be for extension. In some examples, one or more of radii 140-142 may be one of or may include a plurality of sub-radii.

Radii 140-142 may cause femoral component 100 to have a different axis of rotation for different levels of flexion/extension. In addition, radii 140-142 may cause articular surface 112 to have a different curvature for different levels of flexion/extension, leading to different regions or arc segments 130-131 for different regions of flexion/extension.

In some examples, articular surface 112 may include a deep flexion region 130. In various examples, deep flexion region 130 may be optimized based on an SI height 140 of femoral component 100. In any preceding or subsequent examples, articular surface 112 may have a mid-flexion region 131. In some examples, mid-flexion region 131 may be optimized to have a geometry that corresponds or substantially corresponds to patient native anatomy. In various examples, articular surface 112 may have an extension region 132.

In some examples, deep flexion region 130 may be for knee flexion greater than about 90 degrees. In various examples, deep flexion region 130 may be for knee flexion greater than about 70 degrees, greater than about 80 degrees, greater than about 90 degrees, greater than about 100 degrees, greater than about 110 degrees, greater than about 120 degrees, greater than about 130 degrees, or greater than any value or range between any two of these values (including endpoints).

In some examples, mid-flexion region 131 may be for knee flexion between about 15 degrees and 90 degrees. In various examples, deep flexion region 130 may be for knee flexion between about 5 degrees and about 90 degrees, about 10 degrees and about 90 degrees, about 15 degrees and about 90 degrees, about 20 degrees and about 90 degrees, about 25 degrees and about 90 degrees, about 30 degrees and about 90 degrees, about 15 degrees and about 70 degrees, about 15 degrees and about 80 degrees, about 15 degrees and about 90 degrees, about 15 degrees and about 100 degrees, about 15 degrees and about 110 degrees, or any value or range between any two of these values (including endpoints).

In some examples, extension region 132 may be for knee flexion less than about 15 degrees. In various examples, extension region 132 may be for knee flexion less than about 30 degrees, less than about 20 degrees, less than about 15 degrees, less than about 10 degrees, less than about 5 degrees, or less than any value or range between any two of these values (including endpoints).

As the knee moves between various levels of extension/flexion, an MR femoral implant experiences one or more transitions between the different radii. The stability of conventional femoral implants has been adversely affected by these transitions, for example, due to an abrupt change in articular surface geometry. Accordingly, various examples may include a cutover or transition arranged between regions of articular surface 112 that provides for an efficient and smooth transition between regions. For example, articulate surface 112 may be non-faceted (or substantially non-faceted) without abrupt edges where articulate surface 112 transitions from one region (for instance, mid-flexion) to another region (for instance, deep flexion).

In general, a transition may occur as articular surface 112 transitions from a first radius to a second radius. For example, transition 462 (for instance, where line 406 intersects articular surface 112) may be arranged between mid-flexion region 131 and extension region 132. In another example, transition 460 (for instance, where line 408 intersects articular surface 112) may be arranged between deep flexion region 130 and mid-flexion region 131.

In some examples, femoral component 100 may have an extension dwell location, referred to as AP dwell or AP Dwell Location 450. In general, the term AP Dwell may refer to the extension dwell location as measured in an anterioposterior (AP) direction as measured from the posterior-most point of the implant 460, between lines A and B, which run through a first point 460 (e.g., a most-posterior point) on articular surface 112 and a second point 461 at a dwell point (for instance, a lowest SI point on articular surface 112 or a point where the femur (or femoral component) rests on the tibia (or tibial component) when the patient is in a standing position), respectively. For example, AP dwell length 450 may be a length between a line drawn vertically through the lowest point on articular surface 112 (for instance, point 461) and a line drawn vertically through the most posterior point 460.

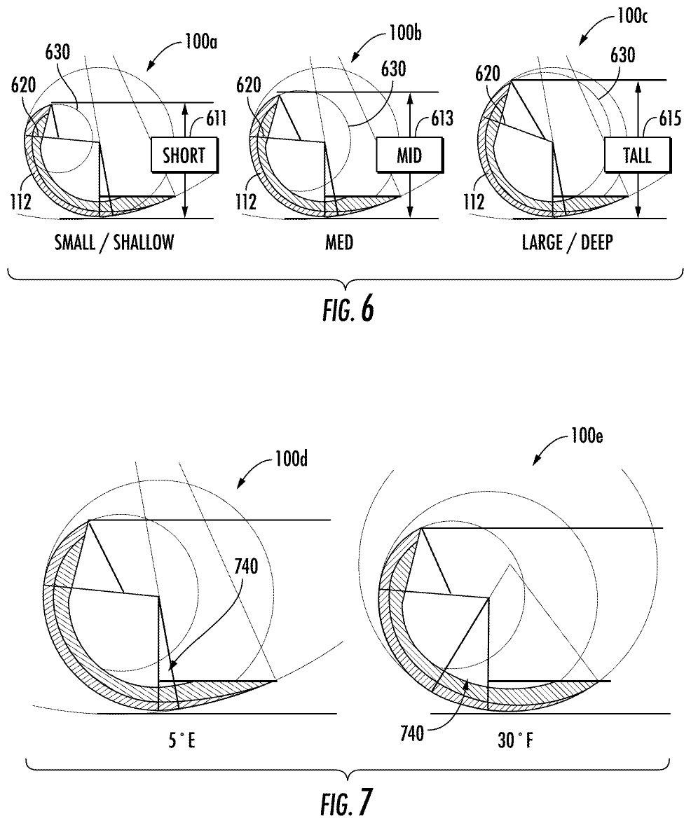

FIG. 5 depicts diagrams of variability boundaries of an illustrative example of a femoral component in accordance with the present disclosure. A femoral component 100 according to some examples may be formed with various variability boundaries or configurations. In general, the different configurations may provide different stability and/or kinematic performance characteristics.

For example, as shown in panel 520, femoral component 100 may be configured with different extension cutovers or transitions 511a, 511b (for instance, one or more points, arcs, and/or the like between where lines 570 and 571 intersect articular surface 112). For example, transitions 511a, 511b may be a transition between an extension region and a mid-flexion region. Referring to panel 521, femoral component 100 may be configured with different flexion cutovers or transitions 513 (for example, one or more points, arcs, and/or the like between where lines 572 and 573 intersect articular surface 112), for instance, between a mid-flexion region and a deep flexion region. Referring to panel 522, femoral component 100 may be configured based on, among other things, a relationship 515 between a mid-flexion region and an AP dwell (see, for example, FIG. 8).

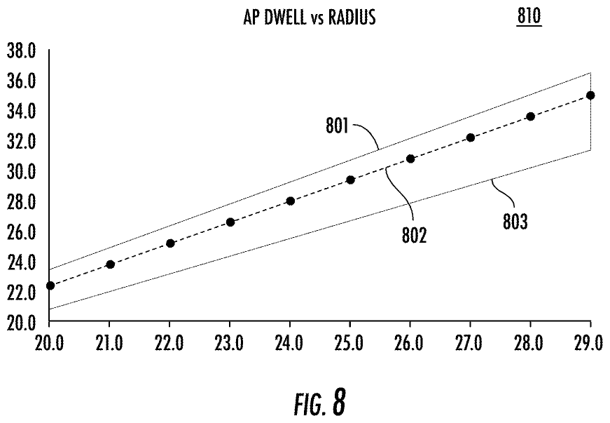

FIG. 6 depicts diagrams of various deep flexion curve transition posterior radius dimensions of an illustrative example of a femoral component in accordance with the present disclosure. As shown in FIG. 6, a femoral component according to some examples, such as femoral components 100a-c, may be configured with a variable deep flexion curve transition 620 (for example, where line 620 intersects articular surface 112), posterior radius 630 properties (for example, implant height, aMCL strain, anterior translation, AP dwell, and/or the like), and/or a relationship therebetween (for instance, a ratio between flexion curve transition 620 and posterior radius 630 properties). In some examples, flexion curve transition 620 and/or posterior radius 630 properties (and/or a relationship therebetween) may be based on an implant (or SI) height of femoral component 100, such as a short (100a), medium (100b), or tall (100c) variations. Different configurations (for instance, configurations 600a-c) may provide for different implant properties, such as aMCL strain, anterior translation, allowable variation, and/or the like.

In various examples, flexion curve transition 620 may be quantified based on degrees of flexion/extension. In various examples, flexion curve transition 620 may be between about 85 degrees of flexion to about 110 degrees of flexion. In some examples, flexion curve transition 620 may be at about 70 degrees of flexion, about 75 degrees of flexion, about 80 degrees of flexion, about 85 degrees of flexion, about 90 degrees of flexion, about 95 degrees of flexion, about 100 degrees of flexion, about 105 degrees of flexion, about 110 degrees of flexion, about 115 degrees of flexion, about 120 degrees of flexion, or any value or range between any two of these values (including endpoints).

FIG. 7 depicts diagrams of various extension curve transition dimensions of an illustrative example of a femoral component in accordance with the present disclosure. As shown in FIG. 7, a femoral component according to some examples, such as femoral components 100d, 100e, may be configured with different extension curve transitions 740. In various examples, extension curve transitions 740 may be quantified based on degrees of flexion/extension. In various examples, an extension curve transition 740 may be between about 5 degrees of extension to about 30 degrees of flexion. In some examples, extension curve transition 740 may be about 15 degrees of extension, about 10 degrees of extension, about 5 degrees of extension, about 0 degrees of flexion/extension, about 5 degrees of flexion, about 10 degrees of flexion, about 15 degrees of flexion, about 20 degrees of flexion, about 25 degrees of flexion, about 30 degrees of flexion, about 35 degrees of flexion, about 40 degrees of flexion, and any value or range between any two of these values (including endpoints). Different configurations (for instance, configurations 100d, 100e) may provide for different implant properties, such as aMCL strain, extension dwell, allowable variation, and/or the like.

Configuration properties of femoral components according to some examples are not limited to the variable configurations depicted in FIG. 5-7. For example, femoral components according to some examples may be configured with more, fewer, and/or different configuration variables and/or combinations thereof. Examples are not limited in this context.

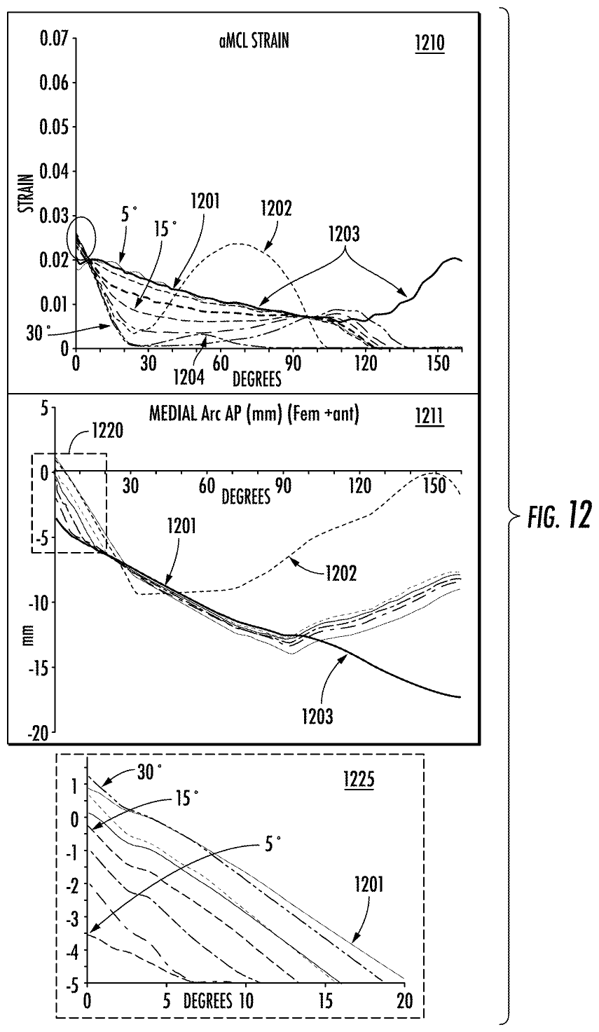

FIG. 8 depicts anteroposterior (AP) and radius relationship information associated with some examples of a femoral component in accordance with the present disclosure. In some examples, a femoral component may be configured according to a relationship between the mid-flexion radius (MFR) and the AP dwell (a “mid-flexion radius/AP dwell relationship” or “MFR/AP relationship”). Graph 810 depicts MFR/AP relationship information 801-803 for femoral components according to some examples. In general, line 801 may represent a maximum MFR/AP relationship value, line 802 may represent a non-limiting example of an optimal MFR/AP relationship according to some examples, and line 803 may represent a minimum MFR/AP relationship value. In general, in some examples, the lines or limits 801 and 803 may indicate the limits of the geometry that is represented in the data in FIGS. 11 and 12. For example, performance data of the extremes of the MFR, cutover angles, AP dwell, SI height, etc. may indicate an “optimal” zone, as presented in FIGS. 11 and 12, with 802 being the ideal or most optimal.

In some examples, the MFR/AP relationship may be determined according to the following:

$$AP dwell = 〈 value 1 〉 ( mid - flexion radius ) - 〈 value 2 〉 .$$

In various examples, value 1 may represent an MFR factor and value 2 may represent an offset value. In some examples, value 1 may be about 1.0 to about 2.0. In some examples, value 2 may be about 2.0 to about 7.0. In any preceding or subsequent examples, value 1 may be about 0.5, about 1.0, about 1.5, about 2.0, about 2.5, about 3.0, about 5.0, about 6.0, about 7.0, about 8.0, and any value or range between any two of these values (including endpoints). In any preceding or subsequent examples, value 2 may be about 0.5, about 1.0, about 1.5, about 2.0, about 2.5, about 3.0, about 5.0, about 6.0, about 7.0, about 8.0, about 10.0 and any value or range between any two of these values (including endpoints).

In various examples, the maximum MFR/AP relationship (line 801) (e.g., upper limit or boundary) may be determined according to the following: AP dwell=1.4493 (mid-flexion radius)−5.4581.

$$AP dwell = 1.4493 ( mid - flexion radius ) - 5.4581 .$$

In some examples, the minimum MFR/AP relationship (line 803) (e.g., lower limit or boundary) may be determined according to the following:

$$AP dwell = 1.1636 ( mid - flexion radius ) - 2.456 1 .$$

In some examples, the example MFR/AP relationship (line 802) may be determined according to the following:

$$AP dwell = 1.4015 ( mid - flexion radius ) - 5.6611 .$$

In some examples, MFR/AP relationship 802 may be an optimal MFR/AP relationship. In various example, lines 801 and 803 may be bounding curves (upper limit or boundary and lower limit or boundary) for an optimal MFR/AP relationship. For example, an optimal MFR/AP relationship may be a relationship between two bounding curves: AP dwellsoi=1.4493 (mid-flexion radius)−5.4581 and AP dwell₈₀₃=1.1636 (mid-flexion radius)−2.4561.

FIGS. 9-12 depict kinematic information associated with some examples of a femoral component in accordance with the present disclosure.

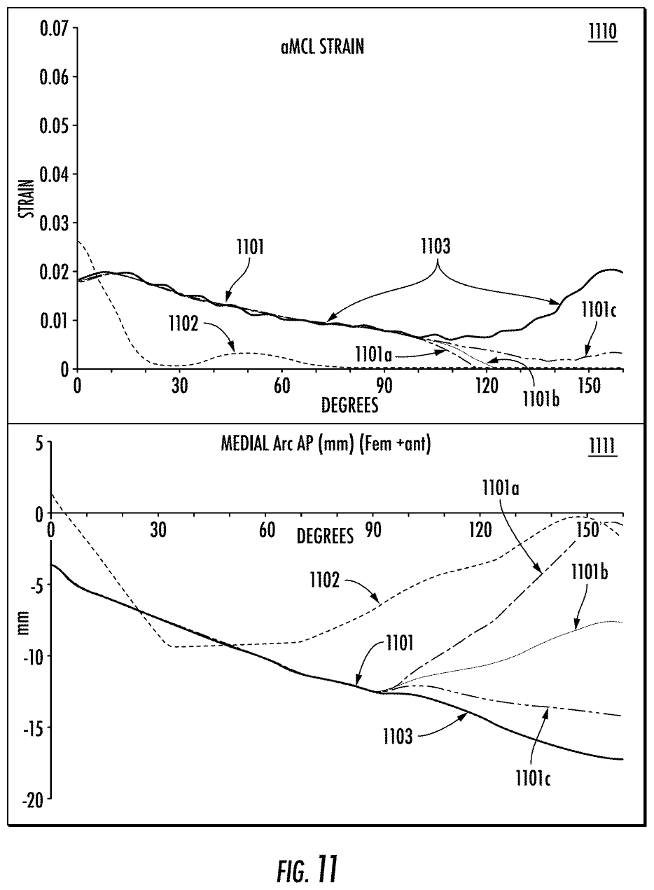

Referring to FIG. 9, therein is depicted graph 910 of MCL strain versus flexion angle. Line 901 represents data for a femoral implant configured according to some examples. Line 902 represents data for a conventional spherical femoral implant and line 903 represents data for a conventional femoral implant. Referring to FIG. 10, therein is depicted graph 1010 of medial arc AP versus flexion angle. In general, the medial arc AP may refer to the tibiofemoral contact location in an anterior-posterior direction, for example, measured from a reference location “0” (y-axis) vs. flexion angle (x-axis), where standing position/terminal extension=flexion angle 0. Line 1001 represents data for a femoral implant configured according to some examples. Line 1003 represents data for a conventional spherical femoral implant and line 1002 represents data for a conventional femoral implant. As indicated by the information graphed in FIGS. 9 and 10, a femoral component configured according to some examples may provide improved kinematic performance compared with conventional knee implant systems.

Referring to FIG. 11, therein is depicted graphs 1110 and 1111 depicting information associated with deep flexion curve variation for MCL strain versus flexion angle and medial arc AP versus flexion angle, respectively. Line 1101 represents data for a femoral implant configured according to some examples for small (1101a), medium (1101b), and large (1101c). Line 1103 represents data for a conventional spherical femoral implant and line 1102 represents data for a conventional femoral implant. As indicated by the information graphed in FIG. 11, a femoral component configured according to some examples may provide improved kinematic performance compared with conventional knee implant systems.

Referring to FIG. 12, therein is depicted graphs 1210 and 1212 depicting information associated with extension flexion curve variation for MCL strain versus flexion angle and medial arc AP versus flexion angle, respectively. Panel 1225 provides a more detailed view of section 1220 of graph 1211. Lines 1201 represents data for a femoral implant configured according to some examples for angles ranging from 5 degrees to 30 degrees. Lines 1202 and 1204 represent data for a conventional femoral implant and line 1203 represents data for a conventional spherical femoral implant. As indicated by the information graphed in FIG. 12, a femoral component configured according to some examples may provide improved kinematic performance compared with conventional knee implant systems.

As shown in FIGS. 9-12, femoral components configured according to some examples may demonstrate improved performance, for example, as measured via aMCL strain and/or medial arc AP, over various ranges of flexion/extension compared with conventional femoral components.

Knee implant systems having a femoral component configured according to some examples may be designed without the compromises made in traditional implant designs such as artificial bone facing shapes to match traditional instrumentation, features such as keels to target implant strength which diminishes bone strength, changes to the articular surface away from the native bony surface to improve ease of implantation, and/or offering a symmetric implant to reduce complexity of implantation or for inventory control. In contrast, femoral component configured according to some examples optimizes all of these features without adding surgical complexity, for example, due to the aid of handheld robotics and/or computer-aided surgical systems.

In various examples, a femoral component configured according to some examples could be developed as a TKA or bi-condylar implant. A femoral component configured according to some examples could feature additional strength features such as a bulbous section that would not create similar stress concentrations as an implant with a keel. A femoral component configured according to some examples could be of similar articular construction but with additional planar preparation to ease implantation with manual instruments. A femoral component configured according to some examples could be constructed with a curved interior surface that matches a standard tool such as a spherical mill, whereby the interior surface does not follow the articular surface with a 3D shape. The articular surface could feature a similar A-P set of curves excluding the spherical cross section in mid-flexion. A femoral component configured according to some examples may include a changeover between the radii in different places (for instance, 10-30 degree range for extension, or 80-120 range for flexion).

The foregoing description has broad application. While the present disclosure refers to certain examples, numerous modifications, alterations, and changes to the described examples are possible without departing from the sphere and scope of the present disclosure, as defined in the appended claim(s). Accordingly, it is intended that the present disclosure not be limited to the described examples. Rather these examples should be considered as illustrative and not restrictive in character. All changes and modifications that come within the spirit of the described examples are to be considered within the scope of the disclosure. The present disclosure should be given the full scope defined by the language of the following claims, and equivalents thereof. The discussion of any example is meant only to be explanatory and is not intended to suggest that the scope of the disclosure, including the claims, is limited to these examples. In other words, while illustrative examples of the disclosure have been described in detail herein, it is to be understood that the inventive concepts may be otherwise variously embodied and employed, and that the appended claims are intended to be construed to include such variations, except as limited by the prior art. Unless otherwise defined, all technical terms used herein have the same meaning as commonly understood by one of ordinary skill in the art to which the disclosure belongs.

Directional terms such as top, bottom, superior, inferior, medial, lateral, anterior, posterior, proximal, distal, upper, lower, upward, downward, left, right, longitudinal, front, back, above, below, vertical, horizontal, radial, axial, clockwise, and counterclockwise) and the like may have been used herein. Such directional references are only used for identification purposes to aid the reader's understanding of the present disclosure. For example, the term “distal” may refer to the end farthest away from the medical professional/operator when introducing a device into a patient, while the term “proximal” may refer to the end closest to the medical professional when introducing a device into a patient. Such directional references do not necessarily create limitations, particularly as to the position, orientation, or use of this disclosure. As such, directional references should not be limited to specific coordinate orientations, distances, or sizes, but are used to describe relative positions referencing particular examples. Such terms are not generally limiting to the scope of the claims made herein. Any examples or feature of any section, portion, or any other component shown or particularly described in relation to various examples of similar sections, portions, or components herein may be interchangeably applied to any other similar examples or feature shown or described herein.

It should be understood that, as described herein, an “example” (such as illustrated in the accompanying Figures) may refer to an illustrative representation of an environment or article or component in which a disclosed concept or feature may be provided or embodied, or to the representation of a manner in which just the concept or feature may be provided or embodied. However, such illustrated examples are to be understood as examples (unless otherwise stated), and other manners of embodying the described concepts or features, such as may be understood by one of ordinary skill in the art upon learning the concepts or features from the present disclosure, are within the scope of the disclosure. Furthermore, references to “one example” of the present disclosure are not intended to be interpreted as excluding the existence of additional examples that also incorporate the recited features.

In addition, it will be appreciated that while the Figures may show one or more examples of concepts or features together in a single example of an environment, article, or component incorporating such concepts or features, such concepts or features are to be understood (unless otherwise specified) as independent of and separate from one another and are shown together for the sake of convenience and without intent to limit to being present or used together. For instance, features illustrated or described as part of one example can be used separately, or with another example to yield a still further configuration. Thus, it is intended that the present subject matter covers such modifications and variations as come within the scope of the appended claims and their equivalents.

As used herein, an element or step recited in the singular and proceeded with the word “a” or “an” should be understood as not excluding plural elements or steps, unless such exclusion is explicitly recited. It will be further understood that the tens “comprises” and/or “comprising,” or “includes” and/or “including” when used herein, specify the presence of stated features, regions, steps, elements and/or components, but do not preclude the presence or addition of one or more other features, regions, integers, steps, operations, elements, components and/or groups thereof.

The phrases “at least one”, “one or more”, and “and/or”, as used herein, are open-ended expressions that are both conjunctive and disjunctive in operation. The terms “a” (or “an”), “one or more” and “at least one” can be used interchangeably herein.

Connection references (e.g., engaged, attached, coupled, connected, and joined) are to be construed broadly and may include intermediate members between a collection of elements and relative to movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority but are used to distinguish one feature from another. The drawings are for purposes of illustration only and the dimensions, positions, order and relative to sizes reflected in the drawings attached hereto may vary.

The foregoing discussion has been presented for purposes of illustration and description and is not intended to limit the disclosure to the form or forms disclosed herein. For example, various features of the disclosure are grouped together in one or more examples or configurations for the purpose of streamlining the disclosure. However, it should be understood that various features of the certain examples or configurations of the disclosure may be combined in alternate examples or configurations. Moreover, the following claims are hereby incorporated into this Detailed Description by this reference, with each claim standing on its own as a separate example of the present disclosure.

1. A multi-radius (MR) femoral component of a knee arthroplasty system, comprising:
an articular surface configured to engage a corresponding tibial component, the articular surface comprising a plurality of regions, each of the plurality of regions associated with a different knee flexion/extension region and defined by one of a plurality of radii of curvature of the articular surface,
wherein the plurality of radii comprises a deep flexion radius, a mid-flexion radius, and an extension radius.

2. The MR femoral component of claim 1, wherein,
the deep flexion radius is configured for greater than about 90 degrees of flexion;
the mid-flexion radius is configured for about 15 degrees to about 90 degrees of flexion; and
the extension radius is configured for less than about 15 degrees of flexion.

3. (canceled)

4. (canceled)

5. The MR femoral component of claim 1, wherein the articular surface is non-faceted at transitions between the plurality of regions.

6. The MR femoral component of claim 1, wherein:
the mid-flexion radius has a circumference of about 15 mm to about 25 mm;
the deep flexion radius has a circumference of about 25 mm to about 50 mm; and
the extension radius has a circumference of about 30 mm to about 70 mm.

7. (canceled)

8. (canceled)

9. The MR femoral component of claim 1, wherein a transition between at least two of the plurality of radii is about 0.5 mm to about 3.0 mm.

10. The MR femoral component of claim 1, wherein at least one of the plurality of radii are formed of a plurality of sub-radii.

11. The MR femoral component of claim 1, wherein the articular surface is configured according to a mid-flexion radius (MFR)/anteroposterior (AP) dwell relationship.

12. The MR femoral component of claim 11, wherein the MFR/AP relationship is defined by: AP dwell=<MFR factor>(MFR)−<offset value>.

13. The MR femoral component of claim 12, wherein:
the MFR factor is a value from about 1.0 to about 2.0; and
the offset value is a value from about 2.0 to about 7.0.

14. (canceled)

15. The MR femoral component of claim 12, wherein an optimal MFR/AP relationship is defined by: AP dwell=<1.4015>(MFR)−<5.6611>.

16. The MR femoral component of claim 12, wherein an optimal MFR/AP relationship is defined within the following first bounding curve and second bounding curve: AP dwell_{first bounding curve}=<1.4493>(MFR)−5.4581 and AP dwell_{second bounding curve}=<1.1636>(MFR)−<2.4561>.

17. The MR femoral component of claim 1, wherein the MR femoral component is non-symmetric to conform to one of a medial condyle or a lateral condyle.

18. The MR femoral component of claim 1, wherein the MR femoral component is a unicompartmental femoral implant.

19. A femoral component of a knee arthroplasty system, comprising:
an articular surface configured to engage a corresponding tibial component, the articular surface comprising a mid-flexion region defined by a mid-flexion radius (MFR),
wherein the articular surface is configured according to a mid-flexion radius (MFR)/anteroposterior (AP) dwell relationship.

20. The femoral component of claim 19, wherein the MFR/AP relationship is defined by: AP dwell=<MFR factor>(MFR)−<offset value>.

21. The femoral component of claim 20, wherein:
the MFR factor is a value from about 1.0 to about 2.0; and
the offset value is a value from about 2.0 to about 7.0.

22. (canceled)

23. The femoral component of claim 20, wherein an optimal MFR/AP relationship is defined by: AP dwell=<1.4015>(MFR)−<5.6611>.

24. The MR femoral component of claim 20, wherein an optimal MFR/AP relationship is defined within the following first bounding curve and second bounding curve: AP dwell_{first bounding curve}=<1.4493>(MFR)−5.4581 and AP dwell_{second bounding curve}=<1.1636>(MFR)−<2.4561>.

25. The femoral component of claim 19, wherein the articular surface comprises a plurality of regions, each of the plurality of regions associated with a different knee flexion/extension region and defined by one of a plurality of radii of curvature of the articular surface,
wherein the plurality of radii comprises a deep flexion radius, the MFR, and an extension radius.

26. The femoral component of claim 25, wherein:
the deep flexion radius is configured for greater than about 90 degrees of flexion;
the mid-flexion radius is configured for about 15 degrees to about 90 degrees of flexion; and
the extension radius is configured for less than about 15 degrees of flexion.

27. (canceled)

28. (canceled)

29. The femoral component of claim 25, wherein the articular surface is non-faceted at transitions between the plurality of regions.

30. The femoral component of claim 25, wherein:
the mid-flexion radius has a circumference of about 15 mm to about 25 mm;
the deep flexion radius has a circumference of about 25 mm to about 50 mm; and
the extension radius has a circumference of about 30 mm to about 70 mm.

31. (canceled)

32. (canceled)

33. The femoral component of claim 25, wherein a transition between at least two of the plurality of radii is about 0.5 mm to about 3.0 mm.

34. The femoral component of claim 25, wherein at least one of the plurality of radii are formed of a plurality of sub-radii.

35. The femoral component of claim 19, wherein the MR femoral component is non-symmetric to conform to one of a medial condyle or a lateral condyle.

36. The femoral component of claim 19, wherein the MR femoral component is a unicompartmental femoral implant.
